(19) United States
(12) Patent Application Publication
(10) Pub. No.: US 2009/0017307 A1
(43) Pub. Date: Jan. 15, 2009

(54) Process for the Preparation of Organic Materials

(75) Inventor: Bansi Lal KAUL, Biel-Benken (CH)

Correspondence Address: NORRIS, MCLAUGHLIN & MARCUS, 875 THIRD AVE, 18TH FLOOR, NEW YORK, NY, 10022, US

(73) Assignee: MCA TECHNOLOGIES, GMBH, Biel-Benken (CH)

(21) Appl. No.: 12/122,051

(22) Filed: May 16, 2008

Related U.S. Application Data
(63) Continuation-in-part of application No. 10/548,331, filed on Sep. 7, 2005.
Application No. 10/548,331 is a continuation of application No. PCT/IB2003/001029, filed on Mar. 20, 2003.

(30) Foreign Application Priority Data
Feb. 20, 2004 (IB) PCT/IB2004/000530

(51) Int. Cl. B32B 27/10; C09K 11/06; C08G 12/24; B32B 27/38; C07D 209/46; C07C 69/76; C07C 229/62; C07D 403/12; C07D 471/04; B32B 27/34; B32B 27/40

(52) U.S. Cl. 428/413; 252/301.35; 524/877; 428/480; 428/479.6; 428/474.4; 428/423.1; 546/70; 548/472; 548/455; 560/54; 560/44

(57) ABSTRACT

The present invention relates to advantageous processes for the manufacture of organic pigments and their precursors. The invention particularly relates to reactions carried out in an “All In One Reactor”® (Draiswerke GmbH, Germany), a kneader like the TurbuKneader® of the same company, a paddle dryer like the Turbudry® of the same company or a related system and thereby submitting the reaction mixtures to enhanced driving power as expressed by a Froude number >1, the reaction mixture being caused to react in high concentrations at elevated temperature.

This is a continuation in part application of U.S. Ser. No. 10/548,331 filed on Sep. 7, 2005 under 35 USC 111(a) of PCT/IB03/01029 filed Mar. 20, 2003 and of U.S. Ser. No. 11/939,945 filed on Nov. 14, 2007 which is a continuation application of continuation in part application U.S. Ser. No. 11/521,194 filed on Sep. 13, 2006 under 35 USC 111(a) of PCT/IB/2004/000530 filed Feb. 20, 2004.

The present invention relates to a particularly advantageous process for preparing of fluorescent and non-fluorescent pigments derived from polymer soluble colorants and/or optical whiteners, for the preparation of quinacridone pigments, isoindolinone pigments, isoindoline pigments, quinophthalone pigments, and the precursors thereof, to the products obtained by such process and to their use as valuable pigments.

The present invention particularly provides a process for preparing the said pigments and their corresponding precursors in a special reactor enabling use of much lesser quantities of solvents and reagents in the synthesis, and particularly the lesser amount of dehydrating agent in the cyclisation process of quinacridone pigments than used in standard batch methods. Use of such smaller quantities of solvents and reagents, particularly the dehydrating agents in the quinacridone cyclisation process, produce high viscosities and are technically unmanageable in state-of-the-art reactors. Use of smaller quantities of solvents and dehydrating agent according to the present invention not only ensures better economy but also better ecology in manufacturing.

### BACKGROUND OF THE INVENTION

Fluorescent pigments are basically the solutions of organic fluorescent dyes in clear and colourless polymeric resins. Typically, a polymer is colored with a fluorescent dye preferably during the condensation/polymerisation process. The resulting colored resin is usually clear, brittle and friable. It is pulverised into a fine powder, which is the final fluorescent pigment (Charles E. Moore in Pigment Handbook I-H, Wiley N.Y. 1988) Postcuring and chemical modification can improve chemical and solvent resistance of such pigments (U.S. Pat. No. 3,412,036). Most fluorescent pigments are based on toluenesulfonamide-melamine-formaldehyde resin matrix (U.S. Pat. No. 29,838,873). Also claimed are the polyamide-type (U.S. Pat. No. 3,915,884), the polyester-type (U.S. Pat. No. 3,922,232) and the urethane-type (U.S. Pat. No. 3,741,907) dye substrates.

In known processes for the manufacture of fluorescent pigments, these thermoset resins are formed by polycondensation of the above mixture in bulk, in non-continuous batches. Such processes are described e.g. in U.S. Pat. No. 3,939,093, in GB 1,341,602 or in U.S. Pat. No. 3,812,051. On the average the reaction takes 2 hours, per batch, in the reactor. After complete polymerisation a hard, tough solid is obtained. This solid must be taken out of the poly-merisation reactor as blocks. This can prove difficult and it is, therefore, often preferred to complete the reaction by casting the reacting mass, which is still pasty, into troughs and finishing the polymerisation in an oven. The blocks are then crushed and finally micronised. Such conventional processes for the manufacture are also described in Chem. Brit., 335 (1977).

The micronisation of this solid presents some difficulties: it requires a pregrinding before a fine microniser is fed, it being necessary for the two items of equipment to be cleaned after each batch. The U.S. Pat. No. 3,972,849 proposes the use of known grinding equipment, such as a ball mill.

The inconvenience of the conventional manufacturing processes and the disadvantages of the pigment particles obtained by these processes have led some manufacturers, on the other hand, to prefer the manufacture and the use of pigments based on thermoplastic resins whenever a high solvent and temperature resistance is not essential. U.S. Pat. No. 2,809,954, GB 869,801 and GB 980,583 describe the synthesis of pigments based on thermoplastic resins. These fusible, and hence heat-sensitive, resins do not lend themselves well to simple micronising by milling and hence to the manufacture of pigments of a fine and well-determined particle size. These resins generally require an additional stage of manufacture (dispersion, phase separation) to obtain pigment particles of well-determined particle size, which is described, for example, in U.S. Pat. Nos. 3,642,650 and 3,412,034.

The disadvantages of the two types of processes described above are avoided in the manufacture of amide (urea, melamine, and the like)/formaldehyde condensates of low molecular weight or of polyester alkyd resins, wherein to each type of said resins the colorant is fixed by adsorption. Such processes are described for example in GB 748,848, GB 786,678 or in GB 733,856. However, the applications of such pigments are in practice limited to inks and paints, because the colorant molecules are bound to the condensates only by adsorption.

U.S. Pat. No. 5,989,453 describes a process wherein the reactants for the formation of the polycondensation resin and the colorant are introduced continuously into an extruder; the mixture is caused to travel forward in the extruder, at the end of reaction the mixture is withdrawn continuously from the extruder, and is deposited continuously onto a conveyor belt, broken up into thermoset flakes, and cooled, the conveyor belt having means for cooling and means for detaching the flakes from the belt. The process still leads to hard thermoset flakes which are still difficult to grind. Besides that, the unreacted potentially toxic monomers, such as formaldehyde, and low molecular weight condensation products remain occluded into the product. Such toxic components are set free, particularly at elevated temperatures during the actual coloring of plastics therewith, causing environmental, health & safety problems.

Quinacridone pigments are one of the most important groups of high-performance organic pigments used universally for almost all applications of organic pigments.

Processes for the preparation of quinacridone pigments are known. E.g., Edward E. Jaffe in “High Performance Pigments” page 279, Ed. by H. M. Smith, Wiley-VCH Verlag-GmbH, Weinheim Germany, 2002; S. S. Labana and L. L. Labana, “Quinacridones” in Chemical Review, 67, 1-18 (1967), and U.S. Pat. Nos. 3,157,659; 3,256,285; and 3,317,539.

The preferred method of synthesis for the formation of quinacridone pigments involves the formation of a dialkyl succinnoylsuccinate of formula II, wherein R₁ represents an alkyl

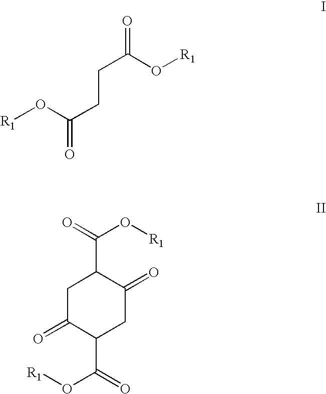

group, from the corresponding dialkyl succinate of formula I either separately (U.S. Pat. Nos. 3,024,268 and 3,045,040) or in situ in the presence of a base in a high boiling inert solvent (U.S. Pat. Nos. 2,821,541 and 3,156,719).

The resulting dialkyl succinoylsuccinate of formula II is in turn reacted with aryl amines, again either separately or in situ, to yield 2,5-di(arylamino)-3,6-dihydroterephthalic acid dialkyl esters of formula III followed by their cyclisation to 6,13-dihydroquinacridones of formula IV and ultimately to the quinacridone pigments of formula VI by oxidation (e.g. U.S. Pat. Nos. 5,659,036; 5,817,817).

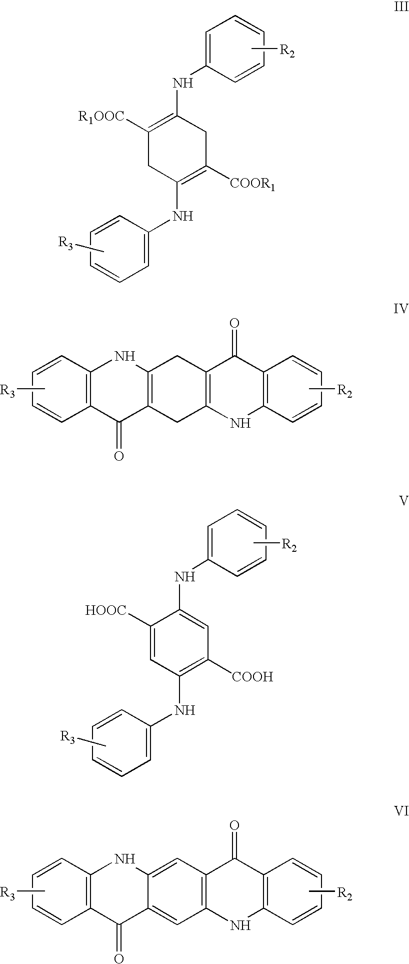

An alternative preferred method for preparing quinacridones involves oxidation or oxidation and hydrolysis of 2,5-di(arylamino)-3,6-dihydroterephthalic acid dialkyl esters of the formula III to the corresponding 2,5-di(arylamino)-terephthalic acid dialkyl esters of the formula VII or 2,5-diarylaminoterephthalic acids of the formula V (e.g. U.S. Pat. Nos. 3,031,501; 4,124,768).

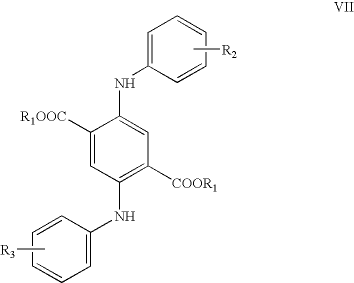

The resulting 2,5-diarylaminoterephthalic acid intermediates of formulae V and VII are then subjected to thermally induced ring closure in the presence of polyphosphoric acid (e.g., U.S. Pat. Nos. 3,257,405; 5,591,258; 6,241,814) or even sulphuric acid (e.g., U.S. Pat. No. 3,200,122 and European Patent Application 863,186). After the ring closure is complete, pouring into a liquid in which the quinacridone is substantially insoluble, usually water and/or an alcohol drowns the melt. To be able to pour out, the reaction needs to be carried out in large quantities of the dehydrating agent, making the process uneconomical and environmentally unfriendly.

The resultant crystalline pigment is then further conditioned by solvent treatment or milling in combination with solvent treatment.

Final particle size of quinacridone pigments can be controlled by the methods used in both synthesis and aftertreatment. For example, quinacridone pigments can be made more transparent by reducing the particle size or more opaque by increasing the particle size. In known methods, particle size is generally controlled during precipitation of the pigment by drowning or during milling or solvent treatment of the crude pigment. Tinctorial strength and transparency of pigments can also be affected by solvent treatment. Aftertreatment steps that manipulate the crude pigments particle size is often referred to as conditioning methods.

Although batch wise processes can produce good quality product, more efficient continuous processes have also been reported for thermally induced ring closure in the presence of polyphosphoric acid for the preparation of quinacridones (see U.S. Pat. No. 6,068,695; WO 02/38680). However, because of the limited dwelling time for the reaction in the continuous process, such processes cannot provide the quality of the products obtained by batch processes without affecting the productivity. Moreover, such continuous processes requiring lesser amounts of reagents have been described only for the ring closure step and not for the preparation of the precursors.

### DESCRIPTION OF THE INVENTION

The objective of the present invention is to manufacture fluorescent and non-fluorescent pigments comprising a white or a colored compound incorporated in a resin which encapsulates, confines and immobilizes the colored compound, which pigments withstand the action of heat or of solvents, while avoiding the disadvantages of the cumbersome processes of the prior art such as multistep processes, the handling, the crushing and the difficulties of micronisation, and especially the elimination of the residual monomers and/or low molecular weight condensation products.

This objective is attained by a process for the manufacture of pigments, comprising a colored compound and/or a fluorescent whitener incorporated in a polycondensation resin by bulk polycondensation of the reaction mixture, wherein the reactants for the formation of said polycondensation resin and the colored or whitening compound are introduced in an apparatus submitting the reaction mixture to enhanced driving power as expressed by a Froude number >1. The mixture is caused to react at a suitable elevated temperature, with or without vacuum, at the same time removing during and/or at the end of the reaction any residual unreacted potentially toxic monomers and/or low molecular weight condensation products formed. The liquid resin composition thus formed is then cooled and allowed to solidify under stirring in the same reactor. Unlike any state-of-the art processes, pigments are thereby directly formed in a broken-up crushed state so that the subsequent micronisation is either not required and/or is facilitated. Furthermore, in situ crushing and micronisation also liberate any occluded monomers and low molecular weight condensation products enabling their easy elimination during the process of the present invention.

A further objective of the present invention is to manufacture organic pigments and their precursors, while avoiding the disadvantages of the cumbersome processes of the prior art such as excessive use of solvents, reagents and even multistep processes.

The present invention therefore provides a process for preparing quinacridone pigments and their precursors inherently bearing the advantages of both batch method, such as quality; and continuous process such as efficiency and better ecology by using smaller amounts of solvents and/or dehydrating agent than used in standard methods, even when such smaller quantities of the solvents and dehydrating agents produce high viscosities. In addition to allowing the use of smaller quantities of solvents and dehydrating agent, which lowers manufacturing costs and reduces environmental impact, the present invention produces quinacridones having characteristics of batch processes.

This objective is attained by a process for the manufacture of quinacridone pigments and/or their precursors in an apparatus by submitting the highly concentrated and viscous reaction mixture to enhanced driving power as expressed by a Froude number >1. The mixture is caused to react at a suitable elevated temperature, with or without vacuum, optionally at the same time removing during and/or at the end of the reaction any volatile by-products formed and smaller amounts of solvents if and when used in the process.

The Froude number Fr is defined by the formula

$$F r = \frac{{v}^{2}}{r \times g}$$

in which v is the velocity of the operative part, r is the radius of the operative part and g is the gravity of the treated materials. Such effect is obtained at overcritical speed >100 r/min and can be achieved independently of the apparatus size. Examples of such apparatus are e.g. “All In One Reactor”® (Draiswerke GmbH, Germany), a kneader like the TurbuKneader® of the same company, a paddle dryer like the Turbudry® of the same company or a related system.

Particularly suitable polycondensation resins to be used according to the instant invention are products which are inelastic, non-fiber-forming and brittle and which consequently may easily be converted into particulate form. The resins should moreover have a relatively high softening point, preferably of more than about 100° C., because otherwise at the temperatures which arise during milling the particles of resin may agglomerate and stick together. The resins should also have little or no solubility in the solvents conventionally used in processing, such as e.g. painters' naphtha, toluene and xylenes and also should not swell in these solvents. Furthermore, the resins should exhibit good transparency and adequate fastness to light. Resins meeting these requirements are generally known, and some of them have already been used for the preparation of daylight fluorescent pigments.

Suitable polycondensation resins are for example those, wherein the reactants for the formation of said polycondensation resins are

(a) at least one component A chosen from aromatic sulfonamides containing 2 hydrogens bonded to the nitrogen of the sulfonamide group,
(b) at least one component B chosen from substances containing 2 or more NH₂ groups, each of the said NH₂ groups being bonded to a carbon, the said carbon being bonded by a double bond to an ═O, ═S or ═N, and
(c) at least one aldehyde component C.

Among the substances capable of forming the component A according to the present invention, there will be mentioned especially benzenesulfonamide and benzenesulfonamide derivatives of general formula:

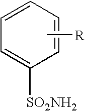

wherein the groups R are hydrogen, alkyl or aryl groups. A particularly preferred substance A is para-toluenesulfonamide, ortho-toluenesulfonamide or mixtures of aromatic sulfonamides, such as mixtures of ortho- and para-toluene-sulfonamide (e.g. a 50:50 mixture of these components), can also be employed and are available on the market. C₁-C₄ alkyl-benzenesulfonamides, e.g. are also available commercially.

Among the substances which can be employed as component B according to the present invention there will be mentioned especially urea (NH₂CONH₂), thiourea (NH₂CSNH₂), guanidine (NH₂)₂C═NH, carbamylurea (C₂H₅N₃O₂), succinamide (C₄H₈N₂O₂), among the noncyclic compounds; among cyclic compounds and more particularly among nitrogenous heterocyclic rings there will be mentioned the molecules containing a plurality of NH₂ groups, each of these groups being bonded to a carbon of a heterocyclic ring, the said carbon being linked by a double bond to a nitrogen of the heterocyclic ring; these heterocyclic rings include the triazole, diazine, triazine and pyrimidine nuclei; there will be mentioned in particular the guanamine derivatives of general formula:

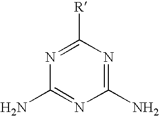

where R′ is hydrogen, an aliphatic radical, an aromatic radical, a saturated or unsaturated cycloaliphatic or alkoxyaryloxy radical. Benzoguanamine may be mentioned among the preferred compounds B.

A compound B which is particularly preferred when it is intended to obtain a thermoset resin is melamine (where R′ is NH₂). Diguanamines and triguanamines (whose synthesis from the corresponding nitriles and from dicyanodiamide is known, furthermore), or mixtures of the above substances can also be employed as component B according to the present invention, as well as the particular triazine compounds described in the U.S. Pat. No. 3,838,063. A certain amount of the component B according to the invention may be replaced by an isocyanuric ring containing compound, such as isocyanuric acid or its alkyl or aryl esters, or its N-alkyl or N-arylderivatives, respectively; pigment compositions comprising such resins are disclosed in U.S. Pat. No. 3,620,993.

The aldehyde or the mixture of aldehydes forming the component C according to the present invention are formaldehyde, acetaldehyde, propionaldehyde (higher aldehydes can be employed but do not offer any particular advantage within the meaning of the present invention). A particularly preferred compound is paraformaldehyde (CH₂O)ₙ, because of its ease of use.

In the process according to the present invention the concentration of component B, which is preferably between approximately 13% and 40% by weight, of the weight of sulfonamide component A in the reaction mixture, can be taken to values which are markedly higher than those employed in the processes for the manufacture of thermoplastic resins. The concentration of component C in the mixture is preferably between 27% and 40% by weight of the sulfonamide.

A harder and more brittle material is thus obtained, which lends itself better to micronisation and which withstands better the action of heat and of solvents. In the case where the amine chosen as component B is melamine, a decrease in the cost of manufacture is also obtained when the proportion of B is increased, given the low cost of this product.

The decrease in the cost of manufacture of the pigments according to the present invention particularly results from the incorporation of many unit operations such as condensation, pouring out of the reaction mass, solidification of the mass, post curing in a separate oven and pre-grinding, into a single operation. Moreover, the poly-condensation reaction is better controlled than in a continuous process.

Further examples of suitable polycondensation resins are i.a. polyamide resins, polyester resins, polycarbonates or polyurethanes. Other suitable resins are polyester/polyamide resins prepared by the reaction of aminoalcohols or aminophenols with polycarboxylic acids, such as the resins described in U.S. Pat. No. 4,975,220.

Particularly suitable polycondensation resins are polyester resins and especially polyamide resins.

Among the preferred resins are crosslinked polyester resins from aromatic polycarboxylic acids or their anhydrides, particularly aromatic dicarboxylic and tricarboxylic acids, such as phthalic acid, isophthalic acid or trimellitic acid, and bifunctional or polyfunctional alcohols, such as ethylene glycol, glycerol, pentaerythritol, trimethylolpropane and neopentyl glycol. Especially preferred are polyester resins from phthalic anhydride and pentaerythritol. Such preferred polyester resins are described for example in DE 961,575 or in the above mentioned U.S. Pat. No. 3,972,849.

Other preferred polyester resins are partially crystalline thermoplastic opaque polyester resins which have a substantial number of amorphous regions and which contain from 35 to 95 equivalent % of crystallinity-producing monomers and from 5 to 65 equivalent % of amorphous producing monomers. Such resins and their use for the preparation of fluorescent pigments are described in EP-A 489,482, especially on page 2, line 57 through page 4, line 40 which are hereby incorporated by reference.

Other preferred polycondensation resins to be prepared and used according to the invention are polyamide resins formed by the reaction of a polyfunctional amine with both a polycarboxylic acid and a monocarboxylic acid, said polyamide being in the molecular weight range from about 400 to about 2500. Such polyamide resins are substantially linear and have at least one carboxy group remaining on the majority of molecules, which permits a thermoplastic resin to be formed which is extremely friable and grindable. The monocarboxylic acid may be added as such or may be formed in situ by reacting a monoamine and a dicarboxylic acid in sufficient quantity to form the desired corresponding monocarboxylic co-condensate to function as a terminator and control the molecular weight of the resin formed. Optionally, whether or not a monocarboxylic acid is added as such, or is formed in situ, a sufficient amount of stabilizing compound of an element from Groups IIA and IIB may be added to further stabilize the pigment. Such preferred polyamide resins are described in the U.S. Pat. No. 3,915,884, which document is incorporated herein by reference.

Preferred polyfunctional amines for the preparation of the instant polyamide resins are polyfunctional, preferably difunctional, primary amines. Particularly preferred are polyfunctional alicyclic primary amines, which form the most friable resins. Most preferred is isophorone diamine (1-amino-3-aminomethyl-3,5,5-trimethyl cyclohexane). Other suitable amines are aliphatic amines having an aromatic ring, such as the m- and p-xylylene diamines; aliphatic polyfunctional primary amines, such as ethylene diamine, diethylene triamine and the like.

Preferred monocarboxylic aromatic acids are benzoic acid and substituted benzoic acids, such as p-toluic, o-toluic, and 4-methoxy benzoic acid.

Preferred aromatic polycarboxylic acids are those which have carboxy groups on noncontiguous carbon atoms, such as isophthalic acid, terephthalic acid, trimesic acid and dicarboxy and tricarboxy naphthalene.

Other preferred polyamide resins are prepared by reaction of a diamine with an excess stoichiometric amount of a diacid. Such resins are described in U.S. Pat. No. 5,094,777, especially in column 2, line 13 through column 4, line 22, which are hereby incorporated by reference.

When a stabilizing compound of elements in Group IIA and Group IIB of the periodic table of elements is used, such compounds should preferably be compatible with the co-condensate and the coloring material. Suitable compounds are e.g. oxides, carbonates or organic acid salts of Group II elements, such as magnesium oxide, magnesium carbonate, zinc oxide, zinc stearate, calcium hydroxide and the like. Zinc oxide is preferred.

Other preferred polycondensation resins to be prepared and used according to the invention are epoxide resins based on bisphenol-A diglycidyl ethers and crosslinked with polyhydric phenols, such as bisphenol-A, with polycarboxylic acid anhydrides, with Lewis acids and particularly with dicyandiamides and related compounds; hybrid polyesters, such as solid saturated polyester resins having free carboxyl groups and being crosslinked with epoxide resins; polyesters, such as solid saturated polyesters having free carboxyl groups and being crosslinked with triglycidylisocyanurate (TGIC); polyurethanes, such as solid saturated polyesters with free hydroxyl groups being crosslinked with polyisocyanates.

The polycondensation resins to be used according to the instant invention may, if appropriate, also contain other additives, such as antioxidants, stabilizing compounds, such as UV-absorbers or light stabilizers as e.g. the hindered amine light stablizers (HALS). Such stabilizers are well known in the art.

The U.S. Pat. No. 3,915,884 and U.S. Pat. No. 5,094,777 disclose, as mentioned above, the preferred polyamides and their use for the manufacture of fluorescent pigments. However, according to that reference the resins are synthesized by a state-of-the-art process. Such a process is characterized by all the disadvantages discussed above for similar prior art processes.

Surprisingly, with the process of the instant invention a much faster, simpler and more convenient synthesis of the above polycondensation resins and of the pigments, particularly fluorescent pigments, is provided.

The pigments according to the invention comprise preferably at least one colored or white compound which is soluble or partially soluble in the resin composition, the preferred concentration of said substance being between 1% and 5% by weight of the pigments. When non-fluorescent dyes, e.g. solvent dyes are used, the preferred concentration may be up to 10% by weight of the pigments.

Colorants and white materials capable of forming a solid solution in a resin are furthermore known and are, in general, listed in the Colour Index. Rhodamines, coumarines, xanthenes, perylenes and naphthalimides will be mentioned by way of example, no limitation being implied. Examples of appropriate colorants are also compounds described in GB 1,341,602, U.S. Pat. No. 3,939,093, U.S. Pat. No. 3,812,051, DE 3,703,495 and in EP-A 422,474.

Other suitable colorants are diketo-pyrrolo-pyrroles (DPP), especially those which are soluble or at least partially soluble in the resins used. Such DPP compounds are known and are described e.g. in U.S. Pat. No. 4,415,685; U.S. Pat. No. 4,810,802; U.S. Pat. No. 4,579,949 and especially in U.S. Pat. No. 4,585,878.

The present invention is particularly adapted to the manufacture of daylight fluorescent pigments, that is to say pigments whose colored composition comprises one or more substances which are fluorescent in daylight and/or optionally one or more common colored substances. However, it is not limited to pigments of this type: by including in a resin according to the invention a compound which does not absorb in the visible but which fluoresces when it is excited by UV radiation, “transparent” pigments are obtained, which can be employed for particular applications such as invisible inks.

The pigments of the invention are suitable for a wide variety of applications, such as paper coating, textile printing, preparation of paints, plastisols, pastes, inks, markers, toners for non-impact printing or cosmetics.

The instant pigments are characterized by high heat stability and high light stability. Therefore they are particularly suitable for the mass coloration of polymers, particularly of those thermoplastic polymers in which the instant pigments can easily be dispersed. Suitable such polymers are e.g. polyesters, polyamides, PVC-polymers, ABS-polymers, styrenics, acrylics or polyurethanes. Particularly suitable polymers are polyolefins, especially polyethylene or polypropylene. It is particularly convenient to use the instant pigments for the preparation of fluorescent polymer, especially polyolefin masterbatches. The instant pigments, particularly those prepared with basic dyes or with solvent dyes, can also advantageously be used in printing inks, e.g. for textile printing.

The present invention also makes it possible to manufacture nonfluorescent colored pigments.

The concentration of the fluorescent substances in the mixture which is to be polycondensed may be adjusted so that the intensity of colour and/or fluorescence are/is maximised. After polycondensation and micronisation the local microconcentration of colored substances dissolved in the polymeric matrix remains constant whatever the subsequent overall dilution of the pigment powder, according to its use.

In the process according to the present invention the polycondensation of the reaction mixture is preferably performed in a temperature range lying between 80° C. and 300° C.

When said polycondensation resin is a polyester resin, a hybrid polyester resin, a polyamide resin, an epoxide resin or a polyurethane resin, the temperature is more preferably between 160° C. and 300° C., especially preferred between 180° C. and 270° C.

When said polycondensation resin is a melamine formaldehyde resin obtained by the polycondensation of components A, B, and C described above, the reaction is preferably performed in a temperature range lying between 100° C. and 250° C.

The products are preferably micronised to a particle size of between 0.5 and 20 μm. The particularly preferred mean particle size is between 1 and 7 μm. An “All In One Reactor”® of Draiswerke Mannheim, Germany has been found particularly suited as a reactor for implementing the process according to the present invention.

According to one particular aspect of the present invention, there is provided a process for the production of dialkyl succinnoylsuccinate of the formula II by the self-condensation of dialkyl succinate of the formula I in the presence of alkali-metal alkylates, without the use of any solvent. The alkali-metal alkylates can be used either as solids, or solutions or dispersions. The alkyl rest of the dialkyl succinate and alkali-metal alkylates used in the present invention is a lower alkyl group having 1 to 4 carbon atoms or a substituted alkyl group having 1 to 4 carbon atoms. Specific examples of the alkyl include methyl, ethyl, isopropyl, n-butyl, iso-butyl, sec-butyl and tert-butyl. Further embodiment of the process is that the alkyl groups of the dialkyl succinate and alkali-metal alkylates need to be identical. The reaction is carried out in an oxygen-free atmosphere at a reaction temperature between 70° C. and 130° C., thereby simultaneously removing the alcohol formed in the reaction, as well as the dispersing liquid medium if and when used.

Further, according to another aspect of the present invention, there is provided a process for the production of 2,5-di(arylamino)-3,6-dihydroterephthalic acid dialkyl esters of the formula III, wherein R₁ is an alkyl group, by a condensation reaction between dialkyl succinoylsuccinate of the formula II and aromatic amino compounds of the formula VIIIa and/or formula VIIIb

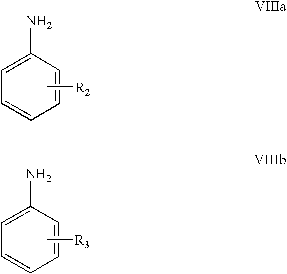

wherein R₂ and R₃ independent of each other are from 0 to 4 substituents selected from the group of F, Cl, Br, I, OH, NO₂, CF₃, an alkyl group having 1 to 4 carbon atoms, a substituted alkyl group having 1 to 4 carbon atoms, an alkoxy group having 1 to 4 carbon atoms, a substituted alkoxy group having 1 to 4 carbon atoms, a phenyl group, a cyclohexyl group, a phenoxy group, COOH, a COOC₁ to C₄ alkyl group, SO₃H, N(CH₃)₂, an N-alkylsulfonamido group such as piperidinomethyl, dimethylaminoethyl, diethylaminoethyl, dimethylaminopropyl, diethylaminopropyl, dibutylaminopropyl, piperidinoethyl, pipecolinoethyl, morpholinoethyl, piperidinopropyl, pipecolinopropyl, diethylaminohexyl, diethylaminobutyl, dimethylaminoamyl, N,N-diethylaminoethyl-N′-laurylamine, 2-ethylhexylaminoethyl, stearylaminoethyl and oleylaminoethyl, a pyridino group, CONH₂ or CON(CH₃)₂, the amount of the aromatic amino compound of the formula VIIIa and/or formula VIIIb being 2.0 to 2.5 mol per mole of the dialkyl succinoylsuccinate of the formula II. The said polycondensation reaction is carried out in the presence, as a catalyst, of an acid such as aromatic sulfonic acids e.g. p-toluenesulfonic acid, hydrochloric acid, sulphuric acid or phosphoric acid in an amount of 0.01 to 0.5 mol per mole of the dialkyl succinoylsuccinate of the formula II. The smaller the amount of water contained in the catalyst, is, the better, since the main reaction proceeds in a dehydration-condensation reaction. The reaction is optionally carried out in the presence, as a dispersing aid, which can be easily removed at the end of the reaction, of an alcohol having 1 to 8 carbon atoms or a glycolmono C₁ to C₄ alkylether or an aliphatic or an aromatic liquid medium such as tetrachloroethane, xylenes, toluene, chlorobenzene, ortho-dichlorobenzene and N-methylpyrrolidone. The reaction is carried out in an oxygen-free atmosphere at a reaction temperature between 80° and 140° C., thereby simultaneously and/or subsequently removing the water formed in the reaction along with the dispersing liquid medium. The preferred liquid mediums are those that can form azeotropes with the water formed in the reaction.

According to a further aspect of the present invention, there is provided a process for producing dihydroquinacridone IV, which comprises mixing the 2,5-di(arylamino)-3,6-dihydroterephthalic acid dialkyl ester III, obtained according to the above process of the present invention for example, with a heating medium commercially available in the trade name of “Dowtherm A” which is a mixture of biphenyl and biphenyl ether, or with any one of alkylnapthalene, N-methylpyrrolidone, dibenzyl ether and t-amyl alcohol, and heating the mixture up to 200° to 350° C. under atmospheric pressure or elevated pressure, whereby the alkyl group and aryl amino group of the ester portion of the 2,5-di(arylamino)-3,6-dihydroterephthalic acid dialkyl ester undergo intramolecular alcohol-elimination and the 2,5-di(arylamino)-3,6-dihydroterephthalic acid dialkyl ester is converted to a corresponding 6,13-dihydroquinacridone which is substituted as required.

The corresponding 6,13-dihydroquinacridone substituted as required is preferably obtained by adding dimethylnaphthalene isomer mixture of which the weight is at the most 2.5 times as large as that of the 2,5-di(arylamino)-3,6-dihydroterephthalic acid dialkyl ester to the above 2,5-di(arylamino)-3,6-dihydroterephthalic acid dialkyl ester and heating the mixture to 200° to 350° C., either under atmospheric pressure or under elevated pressure up to 10 bar in an oxygen-free atmosphere. The alcohol formed is preferably allowed to distil off during the process.

The 6,13-dihydroquinacridones of the formula IV obtained by the above process of the present invention can be converted to a corresponding quinacridone, for example, by oxidizing the 6,13-dihydroquinacridone with an oxidizing agent such as sodium m-nitrobenzenesulfonate, nitrobenzene, nitronaphthalene, nitrobenzenesulfonic acid, nitrobenzenecarboxylic acid, nitrophenol, oxygen or air, in the presence of a mixed solvent of methanol, ethanol, acetone, ethylene glycol or glycol ether with water, in the presence of an alkali, at a high temperature, optionally under elevated pressure, and optionally in the presence of a dispersing agent and a reaction promoter. The oxidation is carried out, for example, with air in the presence of a dispersing agent, preferably an anionic dispersing agent such as a condensate from aromatic sulfonic acid and formaldehyde.

The 2,5-di(arylamino)-3,6-dihydroterephthalic acid dialkyl ester III produced according to the present invention can be converted to a corresponding 2,5-di(arylamino)terephthalic acid V by treating it in a mixed solvent of a solvent with water in the presence of an oxidizing agent and an alkali, at a high temperature, optionally under elevated pressure and optionally in the presence of a dispersing agent and a reaction promoter. The above oxidizing agent includes sodium m-nitrobenzenesulfonate, nitrobenzene, nitronaphthalene, nitrobenzenesulfonic acid and nitrophenol. The above solvent includes methanol, ethanol, acetone, ethylene glycol and glycol ether.

Further, the 2,5-di(arylamino)terephthalic acid V obtained by the above process of the present invention can be converted to a corresponding quinacridone by heating the 2,5-di(arylamino)terephthalic acid up to 100° to 180° C. while mixing it with polyphosphoric acid or polyphosphoric acid of which the weight is 2-3 times as large at the most as that of the 2,5-di(arylamino)terephthalic acid. In this process, the 2,5-di(arylamino)terephthalic acid undergoes an intramolecular-dehydration, ring-closing reaction to be converted to a corresponding quinacridone. Or, the 2,5-di(arylamino)terephthalic acid can be converted to a corresponding quinacridone by a method in which the 2,5-di(arylamino)terephthalic acid V is mixed with a ring-closing agent and an acid catalyst or an organic catalyst in an organic solvent slightly miscible with water and the mixture is heated up to 150° to 210° C., whereby the 2,5-di(arylamino)terephthalic acid undergoes an intramolecular-dehydration, ring-closing reaction to be converted to a corresponding quinacridone. The above ring-closing agent includes nitrobenzene, nitronaphthalene, aniline, phosgene, benzoyl chloride and ethylene glycol. The above acid catalyst includes hydrochloric acid and acetic acid. The above organic catalyst includes quinoline.

The following quinacridones can particularly be synthesized according to the present invention.

quinacridone, 2,9-dichloroquinacridone, 3,10-dichloroquinacridone, 4,11-dichloroquinacridone, 2,3,9,10-tetrachloroquinacridone, 2,4,9,11-tetrachloroquinacridone, 2,9-difluoroquinacridone, 2,9-dibromoquinacridone, 2,9-dimethylquinacridone, 3,10-dimethylquinacridone, 4,11-dimethylquinacridone, 2,4,9,11-tetramethylquinacridone, 2,9-di(tert-butyl)quinacridone, 2,9-dihydroxylquinacridone, 2,9-di(trifluoromethyl)quinacridone, 2,9-dimethoxyquinacridone, 2,9-diethoxyquinacridone, 2,4,9,11-tetramethoxyquinacridone, 2,9-dicarboxylquinacridone, 2,9-dichlorohexylquinacridone, 2,9-diphenylquinacridone, 2,9-di(dimethylamino)quinacridone, 2,9-di(dimethylaminosulfo)quinacridone, 2,9-di(dimethylaminocarbonyl)quinacridone, 3,10-dinitroquinacridone, 2,9-dimethyl-4,11-dichloroquinacridone, 2,9-dimethyl-4,11-dicarboxyquinacridone, and 2,9-dipyridinoquinacridone.

It is also possible to use mixtures of the arylamines VIIIa and VIIIb or the mixtures of 2,5-diarylamino-3,6-dihydroterephthalic acid dialkyl esters III or 2,5-diarylaminoterephthalic acids V in this process. The use of such mixtures provides a particularly advantageous method for obtaining quinacridone solid solutions. Mixtures containing 2,5-diarylaminoterephthalic acid or 2,5-diarylamino-6,13-dihydroterephthalic acid or a derivative thereof in combination with a fully formed quinacridone pigment (generally in crude form) can also be used.

A critical feature of the invention is the inclusion of small quantities of the N-alkylsulfonamido substituted 2,5-diarylamino-3,6-dihydroterephthalic acid dialkyl esters III or 2,5-diarylaminoterephthalic acids V during the ring-closure reaction used to prepare the quinacridone pigment composition. Such pigment compositions exhibit even better pigmentary properties.

Optionally, the small amounts of N-alkylsulfonamido substituted 6,13-dihydroquinacidones or N-alkylsulfonamido substituted quinacidones can be incorporated following the cyclisation process. The amount of such additives may not exceed 5 parts of the final pigment composition in order not to impair its properties.

According to the present invention, there is also provided a process for the production of the isoindolinone compounds of formula IX. E.g., Abul Iqbal et. al. in “High Performance Pigments” page 231, Ed. by H. M. Smith, Wiley-VCH Verlag-GmbH, Weinheim Germany, 2002.

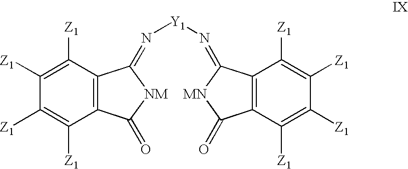

wherein M is a hydrogen or an alkali metal, preferably sodium or potassium, Z₁ halogen or hydrogen, and Y₁ is an aromatic residue of the formula

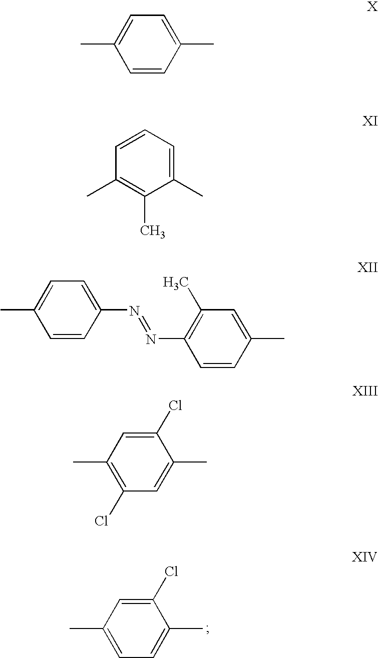

The starting materials used are preferably the isoindolinones of the formula XV produced in situ or separately from the corresponding ester of tetrachloro-o-cyanobenzoic acid of the formula XVI, and the diamines of the formula H₂N—Y₁—NH₂,

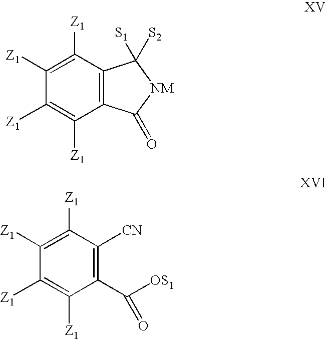

wherein Z₁ has the indicated meaning and S₁ and S₂ denote alkoxy groups. The corresponding ester of tetrachloro-o-cyanobenzoic acid are obtained by the process of DOS 2,301,863.

The following may be mentioned as examples of the isoindolinones of the formula XV:

3,3-dimethoxy-4,5,6,7-tetrachloro-isoindolin-1-one, 3,3-dimethoxy-4,5,6,7-tetrabromo-isoindolin-1-one,

The diamines H₂N—Y₁—NH₂ used are preferably:

p-phenylenediamine, 2-chloro-p-phenylenediamine and 2,6-diaminotoluene.

If alkali metal salts of the 3,3-dialkoxy-4,5,6,7-tetrachloroisoindolin-1-ones XV are used as starting materials, water-miscible organic solvents, for example lower aliphatic alcohols, such as lower alkanols, for example methanol, isopropanol or butanol, lower cyclic ethers, such as dioxane, ethylene glycol monomethyl ether or lower aliphatic ketones, such as acetone, are used with advantage. In these cases, the condensation takes place even at relatively low temperatures from 0 to 20° C. The reaction is advantageously carried out in the presence of agents, which bind bases; as examples of such agents there should be mentioned lower fatty acids, which then simultaneously act as solvents, and especially acetic acid.

According to this invention, there is also provided an advantageous process for the production of the isoindoline compound of the formula XVII. E.g., Volker Radtke et. al. in “High Performance Pigments” page 211, Ed. by H. M. Smith, Wiley-VCH Verlag-GmbH, Weinheim Germany, 2002

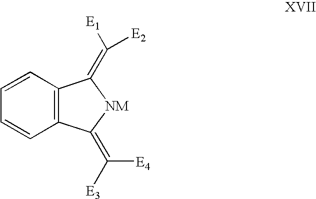

wherein E₁ through E₄ represent CN, CONH-alkyl or CONH-aryl. E₁/E₂ and E₃/E₄ can also be members of a mono- and poly-heterocyclic ring systems or combinations thereof. Examples of such compounds are the pigments and derivatives of: C.I. Pigment Yellow 139, C.I. Pigment Yellow 185, C.I. Pigment Orange 66, C.I. Pigment Orange 69, C.I. Pigment Red 260, C.I. Pigment Brown 38.

The present invention also provides an advantageous process for the production of the quinophthalone compound of the formula XVIII, wherein Y represents a hydrogen or a halogen atom. E.g., Volker Radtke in “High Performance Pigments” page 307, Ed. by H. M. Smith, Wiley-VCH Verlag-GmbH, Weinheim Germany, 2002.

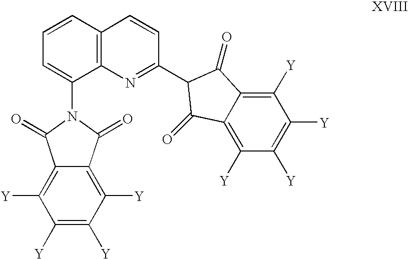

The compounds of the formula XVIII are obtained by the reaction between the 8-aminoquinaldine of formula XIX and the aryldicarboxylic anhydride of the formula XX

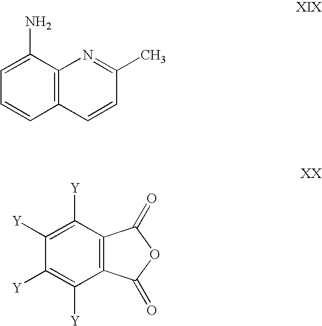

The reaction for the production of quinophthalones may be carried out in the absence of solvent. Generally, however, it is performed in the presence of a solvent. In the process of the present invention, however, the amount of solvent used is considerably less. Useful solvents are organic solvents inert under the reaction conditions, for example, hydrocarbons such as decaline, tetralin or trimethylbenzene; halogenated hydrocarbons such as dichlorobenzene, trichlorobenzene or chloronaphthalene; nitrated hydrocarbons such as nitrobenzene; ethers such as diphenyl ether; and N-methylpyrrolidone.

The reaction is carried out generally under heat. The heating temperature can be varied over a wide range according, for example, to the types and proportions of the starting materials, or the type of the solvent. Usually, it is 150° to 350° C., preferably 180° to 300° C. The reaction pressure is usually normal atmospheric pressure, but if desired, the reaction may be performed at a reduced or elevated pressure from 0.1 to 10 bar. Within the above temperature range, the reaction ends generally in 2 to 10 hours.

The pigments produced according to the present invention are either directly formed or converted into a finely divided form, for pigmenting high molecular organic material, for example cellulose ethers and cellulose esters, such as ethylcellulose, acetylcellulose and nitrocellulose, polyamides or polyurethanes or polyesters, natural resins or synthetic resins, for example aminoplasts, especially urea-formaldehyde and melamine-formaldehyde resins, alkyd resins, phenoplasts, polycarbonates, polyolefines, such as polystyrene, polyvinyl chloride, polyethylene, polypropylene, polyacrylonitrile and polyacrylic acid esters, thermoplastic or thermosetting acrylic resins, rubber, casein, silicone and silicone resins, individually or as mixtures. It is immaterial whether the high molecular compounds mentioned are in the form of plastic compositions or melts or in the form of spinning solutions, lacquers or printing inks. Depending on the end use, it proves advantageous to employ the new pigments as toners or in the form of preparations.

The characteristics and the advantages of the present invention will be understood better with the aid of the examples below. A range of shades extending from yellow to green and black can be produced by mixing e.g. the following colorants, listed in the Colour Index:

### Solvent Yellow 43

### Solvent Yellow 44 (C.I. No. 56200)

### Solvent Yellow 85

### Solvent Yellow 98

### Solvent Yellow 114

### Solvent Yellow 163

### Solvent Yellow 185

### Solvent Yellow 172

### Solvent Orange 63

### Solvent Red 196

### Solvent Red 197

### Solvent Blue 104

### Solvent Green 7

### Basic Yellow 13

### Basic Yellow 19

### Basic Yellow 40

### Basic Yellow 45

### Basic Red 1 (Rhodamine 6G, C.I. No. 45160)

### Basic Violet 10 (Rhodamine B, C.I. No. 45170)

### Basic Blue 7 (C.I. No. 42595)

### Disperse Yellow 232

### Solvent Blue 104

### Solvent Orange 60

A fluorescent whitening agent may also be used alone or be added to colorants.

There now follows a series of examples that serve to illustrate the invention.

### EXAMPLE 1

### Manufacture of Colored Melamine Formaldehyde Condensation Product

A mixture comprising, by weight, 3420 g of p-toluenesulfonamide, 1500 g of paraformaldehyde, 720 g of melamine and 180 g Solvent Yellow 43 colorant was introduced at 20-25° C. in a 10000 ml “All In One Reactor”® (Drais Mannheim Germany).

Under stirring at 220 rpm (Fr=5.4) and nitrogen flow the mixture was heated to 170° C. within 60 minutes. The temperature was maintained at 170° C. for fifteen minutes. The mixture was cooled under stirring to 70° C. and kept at 70° C. The material started solidifying at 110 to 115° C. The brittle friable material thus formed largely disintegrated into an almost semi-powdery material. The reaction mixture was again heated to 100° C. in 30 minutes and kept at 100° C. for 30 minutes for post curing and the elimination of any residual formaldehyde. The mixture was cooled to 50° C. The material was emptied into a polyethylene sack, tightly fitted to the outlet of the reactor. The resin mass could be pulverised by impact milling to a still finer powder for example as described in example 2.

### EXAMPLE 2

### Micronisation

The material recovered from the reactor as described in example 1 was fed into a mill of the air jet microniser type (Alpine 200 AFG, Augsburg).

The operating conditions were: dry air at 7 bars, room temperature, 25 kg/hour flow rate.

More than 99% of the micronised material was between 0.9 and 14 μm in particle size.

### EXAMPLE 3

According to the processes of example 1 and example 2, a fluorescent pink pigment was prepared from a mixture having 70% by weight of para-toluenesulfonamide, 18% by weight of paraformaldehyde, 9% by weight of melamine (dyed by 1.5% by weight of Basic Red 1 and 1.5% by weight of Basic Violet 10, the amount of colorant being relative to the total mixture).

### EXAMPLE 4

A fluorescent pink pigment composition called masterbatch (cylindrical granulate forms with length: 5 mm-diameter: 2 mm) was obtained by including 35 g of pink fluorescent pigment of example 3 in 65 g of a polyvinyl chloride mixture composed of 55% of polyvinyl chloride, 31% of dioctyl phthalate and 2% of an organo-tin stabilizer, and passing said mixture through an extruder at 125° C. The strands of the colored masterbatch were cooled and cut by state-of the art processes to provide the desired granulates.

### EXAMPLE 5

### Manufacture of Colored Polyester Resin

A mixture comprising, by weight, 2740 g of phthalic anhydride, 1225 g of pentaerythritol and 40 g of Rhodamine B was introduced at 20-25° C. in a 10000 ml “All In One Reactor”® of (Drais Mannheim Germany). Under stirring at 220 rpm (Fr=5.4) and nitrogen flow the mixture was heated to 240° C. within 120 minutes. The temperature was maintained at 240° C. for fifteen minutes. The mixture was cooled to 50° C. and kept at 50° C. The brittle friable material thus formed largely disintegrated into an almost semi-powdery material. The material was emptied into a polyethylene sack, tightly fitted to the outlet of the reactor.

### EXAMPLE 6

### Micronisation

The material recovered from the reactor was fed into a mill of the air jet microniser type. The operating conditions were: dry air at 7 bars, room temperature, 20 kg/hour flow rate. The average particle size of the pigments obtained depended on the flow rate and ranged from 1 to 15 μm for more than 99% of the micronised material.

### EXAMPLE 7

### Manufacture of Colored Polyamide Pigment and its Micronisation

According to the process of examples 5 and 6, a fluorescent yellow pigment was prepared from a mixture having 35.3% by weight of isophorone diamine, 34.5% by weight of isophthalic acid, 25.3% by weight of benzoic acid, 3.3% by weight of zinc oxide and 1.6% by weight of Solvent Yellow 98 (C.I. No. 56238).

### EXAMPLE 8

### Preparation of a Masterbatch

A fluorescent pink pigment composition called masterbatch (cylindrical granulate forms with length: 5 mm-diameter: 2 mm) was obtained by including 30 parts of pink fluorescent polyester pigment of example 5 and micronised as described in example 6 in 70 parts of a polyethylene mixture composed of 64 parts of low density polyethylene, 5 parts of polyethylene wax AC 540® Allied Chemical Co.) and 1 part of zinc stearate, and passing said mixture through an extruder at 155° C. The filaments obtained were cooled to room temperature and granulated by state-of-the-art processes.

### EXAMPLE 9

### Preparation of a Masterbatch

A fluorescent yellow pigment composition called masterbatch was obtained as described in example 8 above by using 30 parts of the yellow fluorescent polyamide pigment prepared in example 7 and micronised as described in example 6.

### EXAMPLE 10

### Manufacture of Polyamide Based Pigment and its Micronisation

According to the process of Examples 5 and 6 a fluorescent pink pigment was prepared from a mixture having 14.9% by weight of benzoic acid, 41.5% by weight of isophorone diamine, 40.5% by weight of isophthalic acid and 3.1% by weight of Rhodamine B (C.I. No. 45170).

### EXAMPLE 11

According to the process of Examples 5 and 6 a fluorescent yellow pigment was prepared from a mixture having 10.5% by weight of benzoic acid, 43.7% by weight of isophorone diamine, 42.7% by weight of isophthalic acid and 3.1% by weight of C.I. Basic Yellow 40.

### EXAMPLE 12

According to the process of examples 5 and 6 a fluorescent pink pigment was prepared from a mixture having 29.2% by weight of ethylene glycol, 69.8% by weight of phthalic anhydride and 1% by weight of Rhodamine B.

### EXAMPLE 13

According to the process of examples 6 and 7 a blue pigment was prepared from a mixture having 14.9% by weight of benzoic acid, 41.5% by weight of isophorone diamine, 40.5% by weight of isophthalic acid and 3.1% by weight of Fliso Blue 630® (BASF AG).

### EXAMPLE 14

2280 g of dimethyl succinnoylsuccinate [formula II, in which R₁═CH₃; 4-cyclohexanedione-2,5-di(carboxylic acid methyl ester)], 1953 g of aniline, 2000 ml of isobutanol, and 40 g p-toluenesulfonic acid were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C. The material was emptied into a polyethylene sack, tightly fitted to the outlet of the reactor;

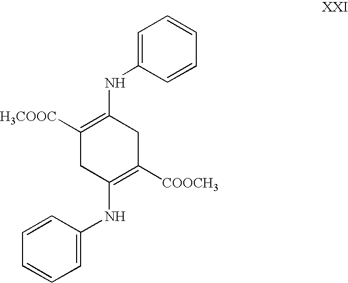

affording 3650 g (96.5% of theory, based on dimethyl succinnoylsuccinate [formula II, in which R₁═CH₃; 4-cyclohexanedione-2,5-di(carboxylic acid methyl ester)] of the compound of the formula XXI. Approximately 250 g of the product were still contained in the reactor to be used in the next batch. The total yield thus corresponded to approximately 3900 g (approximately 99.7% of theory, of approximately

### EXAMPLE 15

Example 14 was repeated except that the aniline was replaced with 2677.5 g of p-chloroaniline, to give 4605 g (99.3% of the theoretical yield) of 2,5-di(p-chloroanilino)-3,6-dihydroterephthalic acid dimethyl ester of formula XXII. The purity thereof was 96.3%.

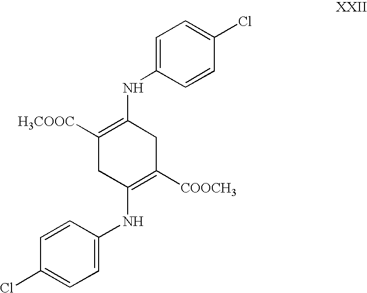

### EXAMPLE 16

Example 14 was repeated except that the aniline was replaced with 2226 g of p-toluidine, to give 4110 g (97.7% of the theoretical yield) of 2,5-di(p-toluidino)-3,6-dihydroterephthalic acid dimethyl ester of the formula XXIII. The purity thereof was 96.3%.

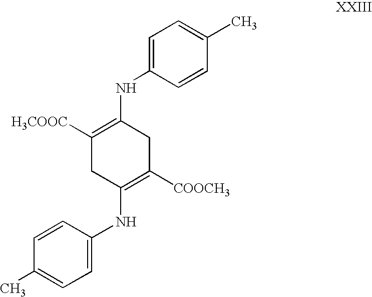

### EXAMPLE 17

1140 g of dimethyl succinnoylsuccinate (formula II, in which R₁═CH₃; 4-cyclohexanedione-2,5-di(carboxylic acid methyl ester), 976.5 g of aniline, 1000 parts of isobutanol, and 25 g of phosphoric acid of 85% concentration were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

For the cyclisation 4000 g polyphosphoric acid (117% phosphoric acid) were now introduced into the reactor and under stirring at 160 rpm (Fr=2.9) and nitrogen flow the mixture was heated to 130° C. within 60 minutes. The temperature was maintained at 130° for 30 minutes. The reaction mass became thick and crumbly. The mixture was cooled to 70° C. Into the reactor were metered over the course of 2 hours 2000 parts of 85% strength phosphoric acid thereby allowing the temperature to rise to 150 degree C. and maintaining it thereat by external cooling during the metering in process. The resultant mass was stirred at 150° C. for one hour and emptied into an HDPE drum. The resultant material was collected by filtration and reslurried in water containing sodium hydroxide (pH greater than 10). The slurry was heated at 90 to 95.degree. C. for one hour, then collected by filtration, washed until free of alkali, and dried to give an 85% yield of dihydroquinacridone of formula XXIV (89% purity).

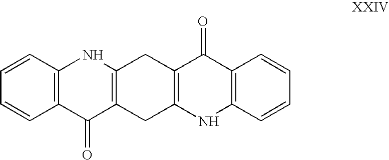

### EXAMPLE 18

1140 g of dimethyl succinnoylsuccinate (formula II, in which R₁═CH₃; 4-cyclohexanedione-2,5-di(carboxylic acid methyl ester), 1113 g of p-toluidine, 1000 parts of isobutanol, and 25 g of phosphoric acid of 85% concentration were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

For the cyclisation 5000 g of a dimethylnaphthalene isomer mixture were now introduced into the reactor and under stirring at 160 rpm (Fr=2.9) and nitrogen flow the mixture was heated to 280° C. within 60 minutes. The temperature was maintained at 280° for 30 minutes, thereby allowing the methanol formed to distil off. The reaction mass became thick and crumbly. The mixture was cooled to 60° C. Into the reactor were metered over the course of 2 hours 2000 parts of methanol. The resultant mass was stirred at 60 degree C. for one hour and emptied into an HDPE drum. The resultant material was collected by filtration and reslurried in water containing sodium hydroxide (pH greater than 10). The slurry was heated at 90 to 95 degree C. for one hour, then collected by filtration, washed until free of alkali, and dried to give an 83% yield of dihydroquinacridone of formula XXV (91% purity).

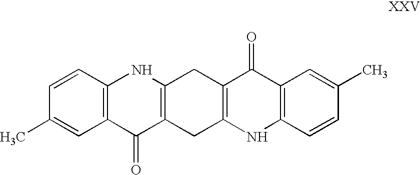

### EXAMPLE 19

4000 g polyphosphoric acid (117% phosphoric acid) and 2000 g of 2,5-dianilinoterephthalic acid (Formula V in which R₂═R₃═H) were placed at 20-25° C. in a 10000 ml “All In One Reactor”® of (Drais Mannheim Germany. Under stirring at 160 rpm (Fr=2.9) and nitrogen flow the mixture was heated to 130° C. within 60 minutes. The temperature was maintained at 130° for 30 minutes. The reaction mass became thick and crumbly. The mixture was cooled to 70° C. Into the reactor were metered over the course of 2 hours 2000 parts of 85% strength phosphoric acid thereby allowing the temperature to rise to 150° C. and maintaining it thereat by external cooling during the metering in process. The resultant mass was stirred at 150° C. for one hour and emptied into an HDPE drum. The resultant material was collected by filtration and reslurried in water containing sodium hydroxide (pH greater than 10). The slurry was heated at 90 to 95 degree C. for one hour, then collected by filtration, washed until free of alkali, and dried to give an 90% yield of the quinacridone of the formula XXVI (93% purity).

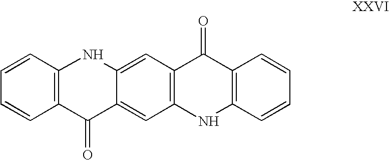

### EXAMPLE 20

5000 g of polyphosphoric acid containing 85.0% P₂O₅ and 1250 parts of 2,5-di-(4-toluidino)terephthalic acid (Formula V wherein R₂═R₃=4-CH₃) were placed at 20-25° C. in a 10000 ml “All In One Reactor”® of (Drais Mannheim Germany). Under stirring at 160 rpm (Fr=2.9) and nitrogen flow the mixture was heated to 120° C. within 30 minutes and maintained at 120° C. for 30 minutes. Thereafter, the mixture was heated to 130 C in 15 minutes and kept at 130 C for one hour. The mixture was cooled to 70° C. followed by metering in of 3000 g isobutanol over a period of 2 hours with external cooling, and thereby allowing the temperature to reach the maximum reflux temperature of isobutanol. The mixture was stirred at reflux temperature for one hour, cooled to 70° C. and emptied into a Steel container. The resulting 2,9-dimethylquinacridone XXVII was collected by filtration and reslurried in water containing sodium hydroxide (pH greater than 10). The slurry was heated at 90 to 95 degree C. for one hour, then collected by filtration, washed until free of alkali, and dried to give an 85% yield of quinacridone of the formula XXVII (95% purity).

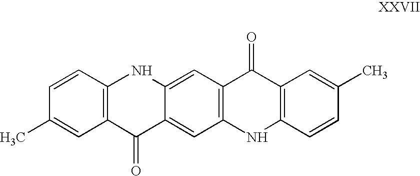

### EXAMPLE 21

2920 g dimethyl succinate (2.0 Mole) and 3600 g 30% sodium methylate solution (2.0 Mole) were placed at 20-25° C. in a 10000 ml “All In One Reactor”® of (Drais Mannheim Germany). Under stirring at 160 rpm (Fr=2.9) and nitrogen flow the mixture was heated to 130° C. within 250 minutes. From 50° C. onwards the reaction mixture became considerably thicker and was finally converted into a paste. From approx. 80° C. onwards a rapid formation of alcohol vapours was observed. The temperature was maintained at 130° C. for three hours, thereby allowing the rest of the alcohol to distil off. The reaction mass became crumbly and finally largely disintegrated into an almost semi-powdery material. The reaction mixture was cooled to 70° C. and 1860 g of aniline were added thereto followed by the addition of 2000 parts of isobutanol. Thereafter 980 g of sulphuric acid of 96% concentration and 25 g of phosphoric acid of 85% concentration were slowly added thereto with external cooling to prevent the temperature to exceed 80 C during the addition. Under stirring at 160 rpm (Fr=2.9) and nitrogen flow the mixture was now heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

The material was emptied into a steel drum affording 4920 g of the product of the formula XXI of 71% purity (92.3% of theory based on 100% material). Approximately 150 g of the product were still contained in the reactor to be used in the next batch.

### EXAMPLE 22

1460 g dimethyl succinate (1.0 Mole) and 1800 g 30% sodium methylate solution (1.0 Mole) were placed at 20-25° C. in a 10000 ml “All In One Reactor”® of (Drais Mannheim Germany). Under stirring at 160 rpm (Fr=2.9) and nitrogen flow the mixture was heated to 130° C. within 250 minutes. From 50° C. onwards the reaction mixture became considerably thicker and was finally converted into a paste. From approx. 80° C. onwards a rapid formation of alcohol vapours was observed. The temperature was maintained at 125° C. for three hours, thereby allowing the rest of the alcohol to distil off. The reaction mass became crumbly and finally largely disintegrated into an almost semi-powdery material. The reaction mixture was cooled to 70° C. and 1060 g of p-toluidine were added thereto followed by the addition of 2000 parts of isobutanol. Thereafter 490 g of sulphuric acid of 96% concentration and 25 g of phosphoric acid of 85% concentration were slowly added thereto with external cooling to prevent the temperature to exceed 80 C during the addition. Under stirring at 160 rpm (Fr=2.9) and nitrogen flow the mixture was now heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

For the cyclisation 4000 g polyphosphoric acid (117% phosphoric acid) were now introduced into the reactor and under stirring at 160 rpm (Fr=2.9) and nitrogen flow the mixture was heated to 130° C. within 60 minutes. The temperature was maintained at 130° for 30 minutes. The reaction mass became thick and crumbly. The mixture was cooled to 70° C. Into the reactor were metered over the course of 2 hours 2000 parts of 85% strength phosphoric acid thereby allowing the temperature to rise to 150 degree C. and maintaining it thereat by external cooling during the metering in process. The resultant mass was stirred at 150 degree C. for one hour and emptied into an HDPE drum. The resultant material was collected by filtration and reslurried in water containing sodium hydroxide (pH greater than 10). The slurry was heated at 90 to 95.degree. C. for one hour, then collected by filtration, washed until free of alkali, and dried to give an 85% yield of 2,9-dimethyldihydroquinacridone of formula XI (89% purity).

### EXAMPLE 23

2280 g of dimethyl succinnoylsuccinate (formula II, in which R₁═CH₃; 4-cyclohexanedione-2,5-di(carboxylic acid methyl ester), 1953 g of aniline, 2000 parts of isobutanol, and 40 g p-toluenesulphonic acid were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 60° C. Into the reactor were now metered 3000 parts of methanol.

Then, 1350 g of sodium m-nitrobenzenesulfonate were added, and immediately thereafter, 2400 parts of a 50% NaOH aqueous solution were added. Then, the mixture was refluxed for 4 hours, acidified with sulphuric acid to PH 3 to give 3565 g (of a theoretical value) of the compound of the formula XXVIII after emptying out filtration and washing water and drying.

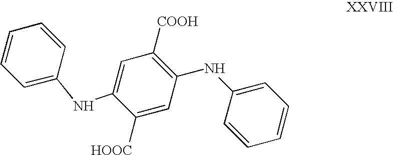

### EXAMPLE 24

1140 g of dimethyl succinnoylsuccinate (formula II, in which R₁═CH₃; 4-cyclohexanedione-2,5-di(carboxylic acid methyl ester), 1113 g of p-toluidine, 1000 parts of isobutanol, and 25 g of phosphoric acid of 85% concentration were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

For the cyclisation 5000 g of a dimethylnaphthalene isomer mixture were now introduced into the reactor and under stirring at 160 rpm (Fr=2.9) and nitrogen flow the mixture was heated to 280° C. within 60 minutes. The temperature was maintained at 280° for 30 minutes, thereby allowing the methanol formed to distil off. The reaction mass became thick and crumbly. The mixture was cooled to 60° C. Into the reactor were metered over the course of 2 hours 2000 parts of methanol.

Then, 1350 g of sodium m-nitrobenzenesulfonate were added, and immediately thereafter, 2400 parts of a 50% NaOH aqueous solution were added. Then, the mixture was refluxed for 4 hours, acidified with sulphuric acid to PH 3 to give 3675 g (of a theoretical value) of the compound of the formula XXVII after emptying out, filtration and washing, water and drying.

### EXAMPLE 25

2000 g of phosphoric acid 85.0% were placed at 20-25° C. in a 10000 ml “All In One Reactor”® of (Drais Mannheim Germany) followed by the slow addition of 2000 g of P₂O₅ under stirring at 160 rpm (Fr=2.9) and letting the temperature to go up to 80° C. Thereafter 1500 parts of 2,5-di-(4-toluidino)terephthalic acid (Formula V wherein R₂═R₃=4-CH₃) were added thereto under stirring at 160 rpm (Fr=2.9) and nitrogen flow. The mixture was now heated to 120° C. within 30 minutes and maintained at 120° C. for 30 minutes. Thereafter, the mixture was heated to 130° C. in 15 minutes and kept at 130° C. for one hour. The mixture was cooled to 70° C. followed by metering in of 3000 ml of isobutanol over a period of 2 hours with external cooling, and thereby allowing the temperature to reach the maximum reflux temperature of isobutanol. The mixture was stirred at reflux temperature for one hour, cooled to 70° C. and emptied into a stainless steel container. The resulting product was collected by filtration and reslurried in water containing sodium hydroxide to pH greater than 10. The slurry was heated at 90° to 95° C. for one hour, then again collected by filtration, washed until free of alkali, and dried to give 1316 g (97% theory) of the compound of the formula XXVII.

### EXAMPLE 26

2000 g of parts of the 6,13-dihydroquinacridone XXIV obtained in Example 17, 4000 ml of methanol and 757 g of sodium methylate were charged into the 10000 ml “All In One Reactor”® of (Drais Mannheim Germany) at 20-25 C. The mixture was heated with stirring to 50° C. and stirred at 50° C. for 15 minutes to form a salt. Thereafter 20000 g of sodium m-nitrobenzenesulfonate was added, and the mixture was refluxed for 4 hours to give 1947 g (98. % of the theoretical yield) of the unsubstituted quinacridone of the formula XXVI

### EXAMPLE 27

700 g of the 6,13-dihydroquinacridone obtained in Example 17 and 2000 ml of methanol were charged to the 10000 ml “All In One Reactor”® of (Drais Mannheim Germany) at 20-25° C., and stirred. 840 g of a 50% NaOH aqueous solution was added, and the mixture was stirred at 30° C. for 30 minutes to form a salt. 910 g of 20% sulphuric acid was added dropwise to hydrolyse the salt, and the reaction mixture was heated to reflux and held at reflux for 1 hour. 700 g of sodium m-nitrobenzenesulfonate was added, and immediately thereafter, 3500 g of a 50% NaOH aqueous solution was added. Then, the mixture was refluxed for 4 hours to give 688 g (99. % of the theoretical yield) of the unsubstituted quinacridone of the formula XXVI.

### EXAMPLE 28

2000 g of parts of 2,5-di(p-toluidino)-3,6-dihydroterephthalic acid dimethyl ester of the formula XXIII obtained in Example 16, 4000 ml of methanol and 757 g of sodium methylate were charged into the 10000 ml “All In One Reactor”® of (Drais Mannheim Germany) at 20-25° C. The mixture was heated with stirring to 50° C. and stirred at 50° C. for 15 minutes to form a salt. Thereafter 1950 g of sodium m-nitrobenzenesulfonate was added, and the mixture was refluxed for 4 hours to give 1950 g (98% of the theoretical yield) of the 2,5-di(p-toluidino)-terephthalic acid dimethyl ester of the formula of the formula XXIX

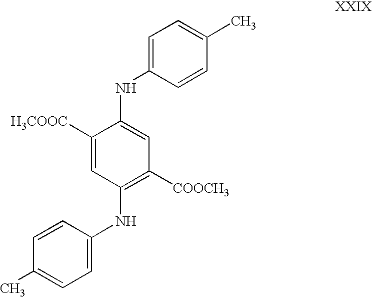

### EXAMPLE 29

2280 g of dimethyl succinnoylsuccinate [formula II, in which R₁═CH₃; 4-cyclohexanedione-2,5-di(carboxylic acid methyl ester)], 2226 g of p-toluidine, 2000 ml of isobutanol, and 40 g p-toluenesulphonic acid were placed at 20-25° C. in a 10000 ml “All In One Reactor”® of (Drais Mannheim Germany). Under stirring at 160 rpm (Fr=2.9) and nitrogen flow the mixture was heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

Into the reactor were now metered 3000 parts of methanol. Then, 2500 g of sodium m-nitrobenzenesulfonate and, immediately thereafter, 1500 g of sodium methylate were added. The mixture was refluxed for 4 hours to give 3860 g (95.5% of the theoretical yield) of 2,5-di(p-toluidino)-terephthalic acid dimethyl ester of the formula XXIX after emptying out, filtration, washing with water and drying.

### EXAMPLE 30

1460 g dimethyl succinate (1.0 Mole) and 1800 g 30% sodium methylate solution (1.0 Mole) were placed at 20-25° C. in a 10000 ml “All In One Reactor”® of (Drais Mannheim Germany). Under stirring at 160 rpm (Fr=2.9) and nitrogen flow the mixture was heated to 130° C. within 250 minutes. From 50° C. onwards the reaction mixture became considerably thicker and was finally converted into a paste. From approx. 80° C. onwards a rapid formation of alcohol vapours was observed. The temperature was maintained at 125° C. for three hours, thereby allowing the rest of the alcohol to distil off. The reaction mass became crumbly and finally largely disintegrated into an almost semi-powdery material. The reaction mixture was cooled to 70° C. and 1060 g of p-toluidine were added thereto followed by the addition of 2000 g of isobutanol. Thereafter 490 g of sulphuric acid of 96% concentration and 30 g p-toluenesulfonic acid were slowly added thereto with external cooling to prevent the temperature to exceed 80° C. during the addition. Under stirring at 160 rpm (Fr=2.9) and nitrogen flow the mixture was now heated to 100° C. within 60 minutes. From 80° C. onwards the reaction mixture became considerably thicker and was finally converted into a paste. The temperature was maintained at 99° to 100° C. for three hours, thereby allowing the mixture of isobutanol and water formed to distil off. The reaction mass became crumbly and finally largely disintegrated into an almost semi-powdery material. The reaction mixture was heated to 120° C. in 30 minutes and kept at 120° C. for 30 minutes under vacuum of 50 mbar. The mixture was cooled to 50° C.

Into the reactor were now metered 3000 ml of methanol. Then, 1500 g of sodium m-nitrobenzenesulfonate and, immediately thereafter, 7500 g of sodium methylate were added. The mixture was refluxed for 4 hours to give 1750 g (86.6% of the theoretical yield) of 2,5-di(p-toluidino)-terephthalic acid dimethyl ester of the formula XXIX after emptying out, filtration, washing with water and drying.

### EXAMPLE 31

Example 30 was repeated except that the aniline was replaced with 1263 g of m-chloroaniline, to give 4605 g (99.3% of the theoretical yield) of 2,5-di(m-chloroanilino)-3,6-dihydroterephthalic acid dimethyl ester of the formula XXX. The purity thereof was 96.3%.

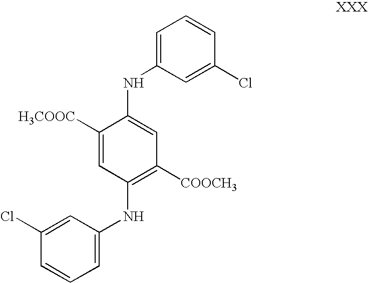

### EXAMPLE 32

2000 g of phosphoric acid 85.0% were placed at 20-25° C. in a 10000 ml “All In One Reactor”® of (Drais Mannheim Germany) followed by the slow addition of 2000 g of P₂O₅ under stirring and letting the temperature to go up to 80° C. Thereafter 1500 parts of 2,5-di-(3-chloroanilino)terephthalic acid dimethyleter XXX were added thereto under stirring at 160 rpm (Fr=2.9) and nitrogen flow. The mixture was now heated to 120° C. within 30 minutes and maintained at 120° C. for 30 minutes. Thereafter, the mixture was heated to 130° C. in 15 minutes and kept at 130° C. for one hour. The mixture was cooled to 70° C. followed by metering in of 3000 ml of isobutanol over a period of 2 hours with external cooling, and thereby allowing the temperature to reach the maximum reflux temperature of isobutanol. The mixture was stirred at reflux temperature for one hour, cooled to 70° C. and emptied into a stainless steel container. The resulting 2,9-dimethylquinacridone of the formula XV was collected by filtration and reslurried in water containing sodium hydroxide to pH greater than 10. The slurry was heated at 90° to 95° C. for one hour, then collected by filtration, washed until free of alkali, and dried to give 1316 g (97% theory) of a mixture of the compounds of the formulas XXXI, XXXII and XXXIII.

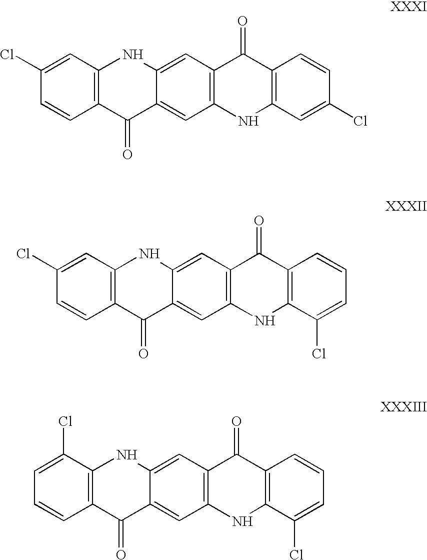

### EXAMPLE 33

4485 g (1.5 Mole) tetrachloro-o-cyanobenzoic acid methyl ester (Formula XV in which Z₁=Cl and S₁═CH₃) and 2400 g 30% sodium methylate solution (1.5 Mole) were placed at 20-25° C. in a 10000 ml “All In One Reactor” of (Drais Mannheim Germany). Under stirring at 160 rpm (Fr=2.9) and nitrogen flow the mixture was heated to 40° C. within 20 minutes. The temperature was maintained at 40° C. for two hours. Thereafter a vacuum of 800 mbars was applied gradually increasing it to 50 mbars, thereby allowing the rest of the alcohol to distil off. The reaction mass became crumbly and finally largely disintegrated into an almost semi-powdery material.

The material was emptied into a steel drum affording approximately 5210 g of the product of the formula XXXIV. Approximately 100 g of the product were still contained in the reactor to be used in next batch.

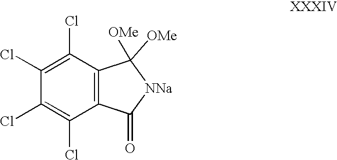

### EXAMPLE 34

2990 g (1.0 Mole) tetrachloro-o-cyanobenzoic acid methyl ester (Formula XV in which Z₁=Cl and S₁═CH₃) and 1800 g 30% sodium methylate solution (1.0 Mole) were placed at 20-25° C. in a 10000 ml “All In One Reactor”® (of (Drais Mannheim Germany). Under stirring at 160 rpm (Fr=2.9) and nitrogen flow the mixture was heated to 40° C. within 20 minutes. The temperature was maintained at 40° C. for two hours. Thereafter 2500 ml of methanol were metered into the reactor followed by the addition of 1026 g (0.95 mole) of p-phenylenediamine. Under stirring at 160 rpm (Fr=2.9) and nitrogen flow the mixture was now heated to 65° C. within 60 minutes. The reaction mixture became considerably thicker and was finally converted into a paste. Thereafter a vacuum of 800 mbars was applied gradually increasing it to 50 mbars, thereby allowing the methanol to distil off. The reaction mass became crumbly and finally largely disintegrated into an almost semi-powdery material.

The material was emptied into a steel drum affording approximately 3210 g of the product of the formula XXXV. Approximately 100 g of the product were still contained in the reactor to be used in next batch.

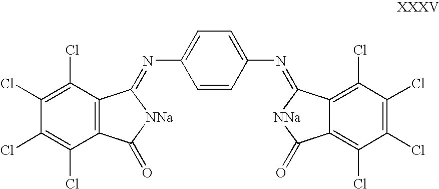

### EXAMPLE 35

2990 g (1.0 Mole)) tetrachloro-o-cyanobenzoic acid methyl ester (Formula XV in which Z₁=Cl and S₁═CH₃) and 1800 g 30% sodium methylate solution (1.0 Mole) were placed at 20-25° C. in a 10000 ml “All In One Reactor”® of (Drais Mannheim Germany). Under stirring at 160 rpm (Fr=2.9) and nitrogen flow the mixture was heated to 40° C. within 20 minutes. The temperature was maintained at 40° C. for two hours. Thereafter 2500 ml of methanol were metered into the reactor followed by the addition of 1026 g (0.95 mole) of p-phenylene-diamine. Under stirring at 160 rpm (Fr=2.9) and nitrogen flow the mixture was now heated to 65° C. within 60 minutes. The reaction mixture became considerably thicker and was finally converted into a paste. The mixture was acidified with 600 g of acetic acid. Thereafter a vacuum of 800 mbar was applied gradually further reducing it to 50 mbars and, thereby allowing the methanol to distil off. The reaction mass became crumbly and finally largely disintegrated into an almost semi-powdery material.

The material was emptied into a steel drum affording approximately 3890 g of the product of the formula XXXVI. Approximately 100 g of the product were still contained in the reactor to be used in next batch.

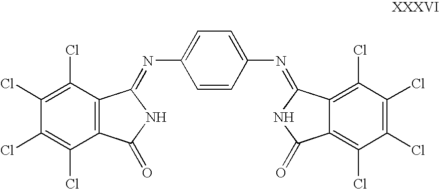

### EXAMPLE 36

1000 g of the material obtained in Example 34 were reslurried in 10000 ml water at 200 to 25° C. containing 200 ml acetic acid. The slurry was heated at 90° to 95° C. for one hour, then collected by filtration, washed until free of acid and salts, and dried to give 850 g the compound of the formula XXXVI.

### EXAMPLE 37

1000 g of the material obtained in Example 35 were reslurried in 10000 ml water at 20° to 25° C. The slurry was heated at 90 to 95° C. for one hour, then collected by filtration, washed until free of acid, and dried to give 790 g the compound of the formula XXXVI

### EXAMPLE 38

1000 g of the material obtained in Example 34 were reslurried in 10000 ml methanol at 200 to 25° C. containing 180 ml acetic acid. The slurry was heated at 650 for four hours, then collected by filtration, washed until free of acid and salts, and dried to give 850 g the compound of the formula XXXVI.

### EXAMPLE 39

2990 g (1.0 Mole) tetrachloro-o-cyanobenzoic acid methyl ester (Formula XV in which Z₁=Cl and S₁═CH₃) and 1800 g 30% sodium methylate solution (1.0 Mole) were placed at 20-25° C. in a 10000 ml “All In One Reactor”® of (Drais Mannheim Germany). Under stirring at 160 rpm (Fr=2.9) and nitrogen flow the mixture was heated to 40° C. within 20 minutes. The temperature was maintained at 40° C. for two hours. Thereafter 2500 ml of o-dichlorobenzene were metered into the reactor followed by the addition of 1160 g (0.95 mole) of 2,6-diaminotoluene. Under stirring at 160 rpm (Fr=2.9) and nitrogen flow the mixture was now heated to 100° C. within 60 minutes and stirred at 100° C. for 4 hours, whilst methanol distils off. Thereafter a vacuum of 800 mbar was applied gradually further reducing it to 50 mbars and, thereby allowing the o-dichlorobenzene to distil off. The reaction mass became crumbly and finally largely disintegrated into an almost semi-powdery material.

The material was emptied into a steel drum affording approximately 3110 g of the product of the formula XXXVII. Approximately 100 g of the product were still contained in the reactor to be used in the next experiment.

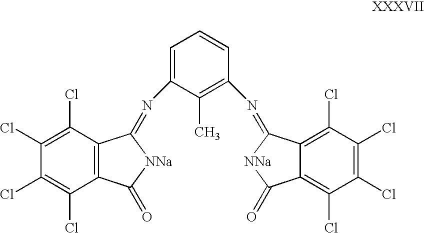

### EXAMPLE 40

2990 g (1.0 Mole) tetrachloro-o-cyanobenzoic acid methyl ester (Formula XV in which Z₁=Cl and S₁═CH₃) and 1800 g 30% sodium methylate solution (1.0 Mole) were placed at 20-25° C. in a 10000 ml “All In One Reactor”® of (Drais Mannheim Germany). Under stirring at 160 rpm (Fr=2.9) and nitrogen flow the mixture was heated to 40° C. within 20 minutes. The temperature was maintained at 40° C. for two hours. Thereafter 2500 ml of o-dichlorobenzene were metered into the reactor followed by the addition of 1160 g (0.95 mole) of 2,6-diaminotoluene. Under stirring at 160 rpm (Fr=2.9) and nitrogen flow the mixture was now heated to 100° C. within 60 minutes and stirred at 100° C. for 4 hours, whilst methanol distils off. The reaction mixture became considerably thicker and was finally converted into a paste. The mixture was acidified with 600 g of acetic acid. Thereafter a vacuum of 800 mbar was applied gradually further reducing it to 50 mbars and, thereby allowing the o-dichloro-benzene to distil off. The reaction mass became crumbly and finally largely disintegrated into an almost semi-powdery material.

The material was emptied into a steel drum affording approximately 4120 g of the product of the formula XXXVIII. Approximately 100 g of the product were still contained in the reactor to be used in the next experiment.

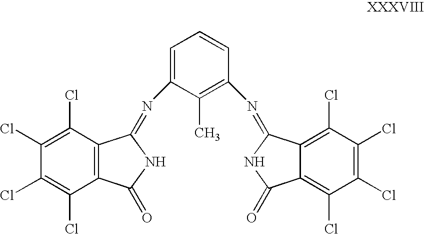

### EXAMPLE 41

1000 g of the material of the formula XXXXVII obtained in Example 39 were reslurried in 10000 ml water at 20° to 25° C. containing 180 ml acetic acid. The slurry was heated at 90° to 95° C. for one hour, then collected by filtration, washed until free of acid and salts, and dried to give 850 g the compound of the formula XXXVIII.

### EXAMPLE 42

1000 g of the material obtained in Example 40 were reslurried in 10000 ml water at 20° to 25° C. The slurry was heated at 90 to 95° C. for four hours, then collected by filtration, washed until free of acid, and dried to give 750 g the compound of the formula XXXVIII.

### EXAMPLE 43

1000 g of the material of the formula XXXXVII obtained in Example 39 were reslurried in 10000 ml methanol at 20° to 25° C. containing 180 ml acetic acid. The slurry was heated at 65° for four hours, then collected by filtration, washed until free of acid and salts, and dried to give 850 g the compound of the formula XXXVIII.

### EXAMPLE 44

3840 g of phthalodinitrile (3.0 Mole) and 50 g 30% sodium methylate solution and 5000 ml of methanol were placed at 30° C. in a 10000 ml “All In One Reactor”® of (Drais Mannheim Germany). Thereafter 600 g of gaseous ammonia were slowly introduced into it over a period of 4 hours thereby maintaining the temperature at 30° C. Thereafter a vacuum of 800 mbars was applied gradually increasing it to 50 mbars, thereby allowing the methanol to distil off. The reaction mass became crumbly and finally largely disintegrated into an almost semi-powdery material.

The material was emptied into a steel drum affording approximately 4250 g of the product of the formula XXXXIX. Approximately 100 g of the product were still contained in the reactor to be used in next batch.

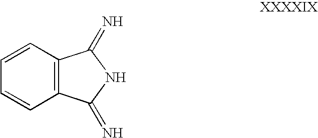

### EXAMPLE 45

1280 g of phthalodinitrile (1 Mole) and 15 g 30% sodium methylate solution and 3000 ml of methanol were placed at 30° C. in a 10000 ml “All In One Reactor”® of (Drais Mannheim Germany). Thereafter 200 g of gaseous ammonia were slowly introduced into it over a period of 4 hours thereby maintaining the temperature at 30° C. Thereafter a vacuum of 800 mbars was applied gradually increasing it to 50 mbars, thereby allowing the methanol to distil off. The reaction mass became crumbly and finally largely disintegrated into an almost semi-powdery material.

Into the reactor were now metered 4000 ml of methanol at 20-25° C. followed by the addition of −2816 g of barbituric acid and 350 ml of glacial acetic acid. Under stirring at 160 rpm (Fr=2.9) and nitrogen flow the mixture was now heated to 65° C. within 60 minutes and maintained at the reflux temperature for 4 hours. Thereafter a vacuum of 800 mbar was applied gradually further reducing it to 50 mbars and, thereby allowing the methanol and excess acetic acid to distil off. The reaction mass became crumbly and finally largely disintegrated into an almost semi-powdery material. The material was emptied into a steel drum affording approximately 3500 g of the product of the formula XL. Approximately 100 g of the product were still contained in the reactor to be used in next batch.

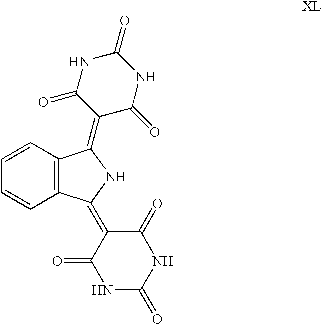

### EXAMPLE 46

1000 g of the material obtained in Example 45 were reslurried in 10000 ml water at 20° to 25° C. The slurry was heated at 90° to 95° C. for five hours, then collected by filtration, washed until free of acid, and dried to give 820 g the compound of the formula XL

### EXAMPLE 47

1450 g (1.0 Mole) of diiminoisoindoline of the formula XXXXIX obtained as described in Example 44, 2816 g of barbituric acid and 4000 ml of methanol and 350 ml of glacial acetic acid were placed at 30° C. in a 10000 ml “All In One Reactor”® of (Drais Mannheim Germany). Under stirring at 160 rpm (Fr=2.9) and nitrogen flow the mixture was now heated to 65° C. within 60 minutes and maintained at the reflux temperature for 4 hours. Thereafter a vacuum of 800 mbar was applied gradually further reducing it to 50 mbars and, thereby allowing the methanol and excess acetic acid distil off. The reaction mass became crumbly and finally largely disintegrated into an almost semi-powdery material. The material was emptied into a steel drum affording approximately 3550 g of the product of the formula XL. Approximately 100 g of the product were still contained in the reactor to be used in next batch.

### EXAMPLE 48

1000 g of the material obtained in Example 45 were reslurried in 10000 ml water at 20° to 25° C. The slurry was heated at 90 to 95° C. for five hours, then collected by filtration, washed until free of acid, and dried to give 820 g of the compound of the formula XL

### EXAMPLE 49

A mixture of 474 g 8-aminoquinaldine, 1888 g tetrachlorophthalic anhydride, 120 g anhydrous zinc chloride and 3000 ml of 1-chloronaphthelene were placed at 20-25° C. in a 10000 ml “All In One Reactor”® of (Drais Mannheim Germany). Under stirring at 160 rpm (Fr=2.9) and nitrogen flow the mixture was heated to 220° C. within 60 minutes and allowed to react for 3 hours at 220° C., steam formed being allowed to escape during the reaction. Thereafter the mixture was cooled to 180° C. and 3000 parts by volume of N-methylpyrrolidone were added thereto. The mixture was stirred at 200° C. for one hour. The product was cooled to 150° C., discharged into a container and separated by filtration. The resulting yellow product was thoroughly washed with 1,000 parts of N-methylpyrrolidone and then with methanol followed by water, and dried to afford 1880 g of a yellow pigment of the formula XLI.

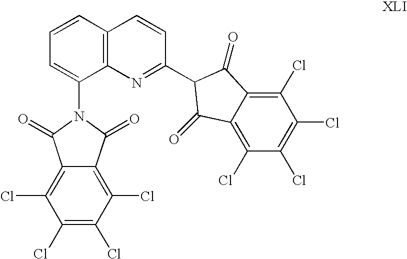

### Application in PVC Masstone

The preparation of a 0.1% colored PVC sheet is performed as follows: 100 parts of clear PVC are mixed with 0.1 part of pigment obtained according to Example 1 for 2 minutes. The mixture is passed between two rollers for 5 minutes, the front roller being heated at 130° C. and the rear roller being heated at 135° C. Then the sheet is pressed under a pressure of 25 tones between two chromium-plated steel plates heated at 165° C., for 5 minutes. The pressed sheet is colored with a red shade.

### Application in PVC White Reduction

The preparation of a 0.1% colored PVC sheet with white is performed as follows: 100 parts of PVC-white (containing 5% TiO) are mixed with 0.1 part of pigment for 2 minutes. The mixture is passed between two rollers for 8 minutes, the front roller being heated at 160° C. and the rear roller being heated at 165° C. Then the sheet is pressed under a pressure of 25 tones between two chromium-plated steel plates heated at 160° C., for 5 minutes.

### Application in Coatings Masstone

The preparation of the alkydmelamine resin coating is performed as follows: 3.6 g of pigment, 26.4 g of clear alkydmelamine paint (35%) and 85 g of glass beads are stirred in a Skandex stirrer for 30 minutes. 30 g of this preparation are mixed with 60 g of clear alkydmelamine paint (55.8%). The dispersion is sprayed on a cardboard sheet, air-dried for 15 minutes and baked at 140° C. in an oven for 30 minutes.

### Application in Coatings White Reduction

The preparation of the alkydmelamine resin coating is performed as follows: 3.6 g of pigment, 26.4 g of clear alkydmelamine paint (35%) and 85 g of glass beads are stirred in a Skandex stirrer for 30 minutes. 7.5 g of this preparation are mixed with 20 g of alkydmelamine white paint (containing 30% TiO). The dispersion is sprayed on a cardboard sheet, air-dried for 15 minutes and baked at 140° C. in an oven for 30 minutes.

### Application in Coatings for Paper

25 parts of blue pigment prepared in example 13, 25 parts of carbital 95, 25 parts of water, 25 parts of latex BASF SD 215® are mixed together. The composition can be used for coating paper.

### Application for Printing Inks

A fluorescent pink ink is prepared from a mixture having 100 parts of binder (Ecocryl® 0254, W. SIPPO Co., Villers Saint Paul, France), 20 parts of a fixer (fixer 99HD®, W. SIPPO Co.), 10 parts of emulsifier (ATEPRINT E9183®, Dr. Th. BÖHME, Germany), 820 parts of water and 20 parts of the pink pigment obtained in example 8. The fluorescent ink is used for application by the screen process (or any similar process) on cotton fabric, which is then heated (dry heat) for 3 minutes at 150° C.

1. A process for the manufacture of pigments, comprising a colored compound and/or a fluorescent whitener incorporated in a polycondensation resin by bulk polycondensation of the reaction mixture, and wherein the reactants for the formation of said polycondensation resin and the colored or whitening compound are introduced in an apparatus submitting the reaction mixture to enhanced driving power as expressed by a Froude number >1, the reaction mixture is caused to react at elevated temperature and the liquid resin composition is cooled and allowed to solidify under continuing driving power.

2. A process according to claim 1, wherein the reaction mixture is treated at a temperature between 80° C. and 300° C.

3. A process according to claim 1, wherein the said coloured compound comprises at least one substance which is fluorescent in daylight and the concentration of the said fluorescent substance is between 0.1% and 10% by weight of the pigments.

4. A process according to claim 1, wherein the said coloured compound comprises at least one substance which is non-fluorescent in daylight and the concentration of the said non-fluorescent substance is between 0.1% and 10% by weight of the pigments.

5. A process according to claim 1, wherein the said materials are micronised to a mean particle size between 0.5 and 20.

6. A process according to claim 1, wherein the said reactors are selected from: “All In One Reactor”® (Draiswerke GmbH, Germany), a kneader like the TurbuKneader® of the same company, a paddle dryer like the Turbudry® of the same company or a related system.

7. A process according to claim 1, wherein the reactants for the formation of said polycondensation resin are
(a) at least one component A which is an aromatic sulfonamide containing 2 hydrogens bonded to the nitrogen of the sulfonamide group,
(b) at least one component B which is a substance containing 2 or more NH₂ groups, each of the said NH₂ groups being bonded to a carbon, the said carbon being bonded by a double bond to an ═O, ═S or ═N, and
(c) at least one aldehyde component C.

8. A process according to claim 7, wherein the concentration of the component B is between 13% and 40% by weight of the component A and the concentration of component C is between 27% and 40% by weight of the component A.

9. A process according to claim 7, wherein the temperature is maintained between 100° C. and 250° C.

10. A process according to claim 1, wherein said polycondensation resin is a polyester resin, a hybrid polyester resin, a polyamide resin, an epoxide resin or a polyurethane resin.

11. A process according to claim 10, wherein said polyester resin is a crosslinked polyester resin from aromatic polycarboxylic acids or their anhydrides and bifunctional or polyfunctional alcohols or wherein said polyester resin is a substantially crystalline thermoplastic opaque polyester resin prepared by reacting mixtures of linear monomers with branched or substituted monomers.

12. A process according to claim 10, wherein said polyamide resin is formed by the reaction of a polyfunctional amine with both a polycarboxylic acid and a monocarboxylic acid, said polyamide being in the molecular weight range from about 400 to about 2500 or wherein said polyamide resin is formed by reacting a diamine with an excess stoichiometric amount of a diacid.

13. A process according to claim 10, wherein the reaction mixture is at a temperature between 160° C. and 300° C.

14. A process according to claim 13, wherein the temperature is maintained between 180° C. and 270° C.

15. A fluorescent or non-fluorescent pigment as prepared by the process of claim 1.

16. A printing ink, paint or lacquer or a paste or plastisol which contains a pigment according to claim 15.

17. A mass-colored plastics material, paper sheet or textile which contains or is coated with a pigment according to claim 15.

18. A process according to claim 5, wherein the said materials are micronised to a mean particle size between 1 and 7 μm.

19. A process for the manufacture of organic pigments selected from the group of: quinacridone pigments, isoindoline pigments, isoindolinone pigments, quinophthalone pigments and of the precursors thereof, which process includes the steps of:
providing reactants for the formation of said materials to a reactor under conditions of elevated temperatures and to enhanced driving power as expressed by a Froude number >1.

20. A process according to claim 19, wherein the said reactor is selected from: an “All In One Reactor”® (ex Draiswerke GmbH, Germany), a kneader, a TurbuKneader® (ex Draiswerke GmbH, Germany) a paddle dryer, and a Turbudry® (ex Draiswerke GmbH, Germany).

21. A process according to claim 19 for the preparation of a compound of the formula VI wherein each R₂ and R₃ independently of the other is a hydrogen, a chlorine, a methyl, a methoxy or an N-alkylsulfonamide group, and/or a mixture thereof.

22. A process according to claim 19 for the preparation of a compound of the formula V wherein each R₂ and R₃ independently of the other is a hydrogen, a chlorine, a methyl, a methoxy or an N-alkylsulfonamide group, and/or a mixture thereof.

23. A process according to claim 19 for the preparation of a compound of the formula IV wherein each R₂ and R₃ independently of the other is a hydrogen, a chlorine, a methyl, a methoxy or an N-alkylsulfonamide group, and/or a mixture thereof

24. A process according to claim 19 for the preparation of a compound of the formula III wherein each R₂ and R₃ independently of the other is a hydrogen, a chlorine, a methyl, a methoxy or an N-alkylsulfonamide group, and/or a mixture thereof.

25. A process according to claim 19 for the preparation of a compound of the formula TI wherein each R₁ is a C₁ to C₈ alkyl radical.

26. A process according to claim 19 for the preparation of a compound of the formula IX

27. A process according to claim 19 for the preparation of a compound of the formula XV

28. A process according to claim 19 for the preparation of a compound of the formula XVII

29. A process according to claim 19 for the preparation of a compound of the formula XVIII.

30. A process according to claim 19 for the preparation of the compound of the formula XXXXIX

31. A process according to claim 19, wherein the elevated temperature is a temperature between 60° C. and 350° C.

32. A pigment as prepared by the process according to claim 19.

33. A process according to claim 19, wherein the reactor is operated under vacuum conditions.

34. A process for the manufacture of organic pigments selected from the group of:
quinacridone pigments, isoindoline pigments, isoindolinone pigments, quinophthalone pigments and of the precursors thereof, which process includes the steps of:
providing reactants for the formation of said materials to a reactor, operating said reactor under conditions of elevated temperatures wherein the reactants are at a temperature of between 60° C. and 350° C., and operating the reactor such that an operative part of the reactor is operated according to the Froude number defined by the formula:
$$F r = \frac{{v}^{2}}{r \cdot g}$$
in which v is the velocity of an operative part of the reactor, r is the radius of the operative part and g is the gravity of the reactants.

35. A process according to claim 34, wherein the reactor is operated under vacuum conditions.

36. A process according to claim 34 wherein the reactor is operated at overcritical speed conditions of >100 r/minute.
